(19) United States
(12) Patent Application Publication
(10) Pub. No.: US 2023/0103151 A1
(43) Pub. Date: Mar. 30, 2023

(54) tRNA OVEREXPRESSION AS A THERAPEUTIC APPROACH FOR CHARCOT-MARIE-TOOTH NEUROPATHY ASSOCIATED WITH MUTATIONS IN tRNA SYNTHETASES

(71) Applicant: Stichting Radboud Universiteit, Nijmegen (NL)

(72) Inventor: Erik Jan Marthe STORKEBAUM, Nijmegen (NL)

(73) Assignee: Stichting Radboud Universiteit, Nijmegen (NL)

(21) Appl. No.: 17/759,574

(22) PCT Filed: Jan. 27, 2021
(86) PCT No.: PCT/NL2021/050048

§ 371 Date: Jul. 27, 2022

(30) Foreign Application Priority Data
Feb. 5, 2020 (NL) 2024840

(51) Int. Cl. C12N 15/10; C12N 15/86; A61P 25/02

(52) U.S. Cl. CPC C12N 15/102; C12N 15/86; A61P 25/02

(57) ABSTRACT

The present invention is in the field of a compound for use as a medicament for treatment of tRNA deficiencies in living cells, a dosage comprising said compound, and an in vivo and in vitro method for treatment of tRNA deficiencies, as well as for prevention, mitigation of symptoms, and regeneration of cells.

### FIELD OF THE INVENTION

The present invention is in the field of a compound for use as a medicament for treatment of tRNA deficiencies in living cells, a dosage comprising said compound, and an in vivo and in vitro method for treatment of tRNA deficiencies, as well as for prevention, mitigation of symptoms, and regeneration of cells.

### BACKGROUND OF THE INVENTION

The present invention is in the field of tRNA deficiencies in living cells, such as a motor or sensory neuron. An example thereof is Charcot-Marie-Tooth disease.

Charcot-Marie-Tooth (CMT) peripheral neuropathy is now an incurable disease characterized by selective degeneration of peripheral motor and sensory axons. In Charcot-Marie-Tooth disease, degeneration of motor and sensory nerves is found to lead to muscle weakness and sensory deficits. CMT is the most common inherited neuromuscular disorder (prevalence: 1:2500), estimated to affect more than 200.000 people in the European Union alone. Traditionally, a distinction can be made between demyelinating forms of CMT (CMT1) and axonal forms (CMT2). More recently, intermediate forms of CMT, with features of both demyelination and axonal degeneration, have been recognized. The molecular mechanisms underlying CMT and the reason why peripheral motor and sensory neurons are selectively affected are poorly understood, and effective drugs are lacking.

Morelli et al. in “Allele-specific RNA interference prevents neuropathy in Charcot-Marie-Tooth disease type 2D mouse models”, Sep. 26, 2019, J Clin Invest. 2019; 129(12):5568-5583 (https://doi.org/10.1172/JCI130600) report Gene therapy approaches being deployed to treat dominantly inherited genetic disorders, caused by heterozygous mutations (in this case in the glycyl-tRNA synthetase), by reducing the expression of mutated genes. However, in mouse models of CMT caused by heterozygous mutations in glycyl-tRNA synthetase it is shown that a microRNA that targets the mutant transcript (=mRNA) encoding glycyl-tRNA synthetase has a therapeutic effect. In other words, they use a microRNA to knock-down the expression of CMT-mutant glycyl-tRNA synthetase (a protein). The efficacy of allele-specific microRNA as a potential therapy for Charcot-Marie-Tooth disease type 2D (CMT2D), caused by dominant mutations in glycyl-tRNA synthetase (GARS) is studied. A de novo mutation inGARS was identified in a patient with a severe peripheral neuropathy, and a mouse model precisely recreating the mutation was produced. These mice developed a neuropathy by 3-4 weeks of age, validating the pathogenicity of the mutation. microRNA sequences targeting mutant Gars mRNA, but not wild-type, were optimized and then packaged into AAV9 for in vivo delivery. This substantially mitigated the neuropathy in mice treated at birth. Delaying treatment until after disease onset showed modest benefit, and this effect is considered to further decrease the longer treatment was delayed. These outcomes were reproduced in a second mouse model of CMT2D using a vector specifically targeting that allele. The effects were dose dependent, and persisted for at least 1 year. These findings demonstrate the feasibility of AAV9-mediated allele-specific knockdown and provide proof of concept for gene therapy approaches for dominant neuromuscular diseases.

The present invention relates to a tRNA overexpression or supplementation as a therapeutic approach for Charcot-Marie-Tooth neuropathy associated with mutations in tRNA synthetases, which overcome one or more of the above disadvantages, without jeopardizing functionality and advantages.

### SUMMARY OF THE INVENTION

The present invention relates in a first aspect to a compound for overexpression of cognate tRNA for use in a medicament for treating a heterozygous mutated cell (i.e. inherited), or for that matter, a disease involving a heterozygous mutated cell, such as an inherited neuromuscular disorder, wherein the compound comprises a transfer RNA, a vector, and optionally a promotor. A very important disadvantage of the approach by Morelli et al. is that the prior art technology only allows allele-specific knock-down if the mutant mRNA differs sufficiently from the wild type mRNA. For the two alleles targeted in this paper, this is indeed the case, as in one allele 12 nucleotides are deleted from the mRNA, and in the other 5 nucleotides are different between WT and mutant transcripts. However, almost all CMT-causing mutations are missense mutations, whereby only a single amino acid in the protein is mutated into another amino acid. This is typically due to a single base pair change in the mRNA. Such a single base pair change is insufficient for specific targeting by a microRNA. The present compound comprises a vector and may therefore also be referred to as a vector compound. The term “compound” is considered to relate to combined parts, in the present case the vector, the transfer RNA, and the optional promotor; in that sense it may also be considered to relate to a “complex”, that is composed of two or more parts. The term “cognate tRNA” is considered to also relate to a tRNA encoding sequence. In an alternative, or in addition also tRNA supplementation may be envisaged. The term “medicament” is considered to relate to a medication, which may also be referred to as “medicine”, “pharmaceutical drug”, or simply “drug”, is a drug used to diagnose, cure, treat, or prevent disease. A drug is e.g. a natural or synthetic substance used in the preparation of said medication. It is found that heterozygous mutations in six distinct tRNA synthetase (aaRS) genes cause CMT. Heterozygous mutations in six distinct genes encoding cytoplasmic aminoacyl tRNA synthetases (aaRSs) are found to cause CMT, namely glycyl- GlyRS), tyrosyl-(TyrRS), alanyl-(AlaRS), histidyl-(HisRS), methionyl-(MetRS), and tryptophanyl (TrpRS)-tRNA synthetase. aaRSs are enzymes that covalently attach amino acids to their cognate tRNAs (tRNA aminoacylation). This reaction constitutes the essential first step of protein biosynthesis. Aminoacylated tRNAs are subsequently transferred to elongation factor eEF1A, which is found to deliver the tRNA to the ribosome for use during protein synthesis/mRNA translation (FIG. 1). The inventors generated Drosophila CMT-aaRS models and used a novel ground-breaking method for in vivo cell-type-specific labelling of newly synthesized proteins to show that impaired protein synthesis may represent a common pathogenic mechanism. Remarkably, overexpression of the cognate tRNA rescued protein synthesis and peripheral neuropathy in Drosophila and mouse models of CMT-aaRS. These data suggested a defect in the transfer of the (aminoacylated) tRNA from the mutant tRNA synthetase to elongation factor eEF1A as the molecular mechanism underlying CMT-aaRS. This can lead to insufficient supply of the cognate aminoacylated tRNA to the ribosome and stalling of the ribosome on cognate codons, resulting in the protein synthesis defect. This detailed molecular working model is now validated and expanded. Furthermore, using in vivo cell-type-specific labelling of newly synthesized proteins in mouse models, the tissue-specificity of CMT-aaRS may be due to more pronounced inhibition of protein synthesis in motor and sensory neurons as compared to other cell types. The therapeutic potential of increasing cognate tRNA levels by synthetic tRNA administration or gene transfer in CMT-aaRS mouse models is evaluated. Therewith provision of alanyl-, glycyl-, tyrosyl-, histidyl-, methionyl- and tryptophanyl-tRNA, respectively, are aimed at with the present vector-[optional promotor]-tRNA compound. Therewith a treatment for this type of nowadays incurable diseases is found, as well as a method for prevention, and for mitigating symptoms thereof.

In a second aspect, the present invention relates to a dosage comprising a compound according to the invention, wherein in a viral gene transfer variant thereof the compound comprises >10¹² vg/kg body mass (body mass typically being 20-100 kg), preferably >10¹³ vg/kg body mass, more preferably >5*10¹³ vg/kg body mass, even more preferably >10¹⁴ vg/kg body mass, such as >2.5*10¹⁴ vg/kg body mass.

In a third aspect, the present invention relates to an in vivo or in vitro method of treating or preventing a heterozygous mutated cell, or preventing symptoms thereof, or for mitigating symptoms thereof, or for regeneration of impaired cells, or for gene therapy, or for RNA therapy, or a combination thereof, comprising providing a dosage according to the invention, and applying the dosage, such as by intrathecal application, and/or by cerebral application, by application to the Peripheral Nervous System, or by systemic application.

In a fourth aspect, the present invention relates to an in vivo or in vitro method of introducing a cognate tRNA or tRNA encoding sequence into a heterozygous mutated cell, comprising providing the heterozygous mutated cell, providing the tRNA or tRNA encoding sequence in a suitable form, wherein the tRNA or tRNA encoding sequence is selected from tRNA^{Ala}, tRNA^{Gly}, tRNA^{Tyr}, tRNA^{His}, tRNA^{Met}, tRNA^{Trp}, and combinations thereof, introducing the tRNA or tRNA encoding sequence into the heterozygous mutated cell. Typically, one tRNA would suffice however, depending on the mutation characteristics of the heterozygous mutated cell. An example of such a tRNA encoding sequence is given in the text file.

The present invention is also subject of a scientific publication

Thereby the present invention provides a solution to one or more of the above-mentioned problems.

Advantages of the present invention are detailed throughout the description.

### DETAILED DESCRIPTION OF THE INVENTION

The present invention relates in a first aspect to a compound for overexpression of cognate tRNA for use in a medicament for treating a heterozygous mutated cell.

In an exemplary embodiment of the present compound the heterozygous mutated cell may be a neuron, such as a motor or sensory neuron, and the treatment may be for treating peripheral neuropathy.

In an exemplary embodiment of the present compound the peripheral neuropathy may be selected from an inherited neuromuscular disorder, such as Charcot-Marie-Tooth peripheral neuropathy, a central nerve system disorder, a brain disorder, a motoric nerve disorder, a sensoric nerve disorder, or a combination thereof.

In an exemplary embodiment of the present compound the vector may be an adeno-associated viral (AAV) vector, preferably an AAV9 (serotype 9) vector.

In an exemplary embodiment of the present compound the promotor may be an RNA polymerase III promotor, preferably a Class I, a Class II, or a Class III, preferably of a small nuclear RNA (snRNA), such as U6 snRNA.

Examples of the present compound are tRNA^{Ala}-AAV, tRNA^{Gly}-AAV, tRNA^{Tyr}-AAV, tRNA^{His}-AAV, tRNA^{Met}-AAV, and tRNA^{Trp}-AAV, such as tRNA^{Ala}-AAV9, tRNA^{Gly}-AAV9, tRNA^{Tyr}-AAV9, tRNA^{His}-AAV9, tRNA^{Met}-AAV9, and tRNA^{Trp}-AAV9, tRNA^{Ala}-Promotor-AAV, tRNA^{Gly}-Promotor-AAV, tRNA^{Tyr}-Promotor-AAV, tRNA^{His}-Promotor-AAV, tRNA^{Met}-Promotor-AAV, and tRNA^{Trp}-Promotor-AAV, such as tRNA^{Ala}-Promotor-AAV9, tRNA^{Gly}-Promotor-AAV9, tRNA^{Tyr}-Promotor-AAV9, tRNA^{His}-Promotor-AAV9, tRNA^{Met}-Promotor-AAV9, and tRNA^{Trp}-Promotor-AAV9, such as tRNA^{Ala}-RNA polymerase III Class I Promotor-AAV, tRNA^{Gly}-RNA polymerase III Class I Promotor-AAV, tRNA^{Tyr}-RNA polymerase III Class I Promotor-AAV, tRNA^{His}-RNA polymerase III Class I Promotor-AAV, tRNA^{Met}-RNA polymerase III Class I Promotor-AAV, and tRNA^{Trp}-RNA polymerase III Class I Promotor-AAV, such as tRNA^{Ala}-RNA polymerase III Class I Promotor-AAV9, tRNA^{Gly}-RNA polymerase III Class I Promotor-AAV9, tRNA^{Tyr}-RNA polymerase III Class I Promotor-AAV9, tRNA^{His}-RNA polymerase III Class I Promotor-AAV9, tRNA^{Met}-RNA polymerase III Class I Promotor-AAV9, and tRNA^{Trp}-RNA polymerase III Class I Promotor-AAV9, such as tRNA^{Ala}-RNA polymerase III Class II Promotor-AAV, tRNA^{Gly}-RNA polymerase III Class II Promotor-AAV, tRNA^{Tyr}-RNA polymerase III Class II Promotor-AAV, tRNA^{His}-RNA polymerase III Class II Promotor-AAV, tRNA^{Met}-RNA polymerase III Class II Promotor-AAV, and tRNA^{Trp}-RNA polymerase III Class II Promotor-AAV, such as tRNA^{Ala}-RNA polymerase III Class II Promotor-AAV9, tRNA^{Gly}-RNA polymerase III Class II Promotor-AAV9, tRNA^{Tyr}-RNA polymerase III Class II Promotor-AAV9, tRNA^{His}-RNA polymerase III Class II Promotor-AAV9, tRNA^{Met}-RNA polymerase III Class II Promotor-AAV9, and tRNA^{Trp}-RNA polymerase III Class II Promotor-AAV9, such as tRNA^{Ala}-RNA polymerase III Class III Promotor-AAV, tRNA^{Gly}-RNA polymerase III Class III Promotor-AAV, tRNA^{Tyr}-RNA polymerase III Class III Promotor-AAV, tRNA^{His}-RNA polymerase III Class III Promotor-AAV, tRNA^{Met}-RNA polymerase III Class III Promotor-AAV, and tRNA^{Trp}-RNA polymerase III Class III Promotor-AAV, such as tRNA^{Ala}-RNA polymerase III Class III Promotor-AAV9, tRNA^{Gly}-RNA polymerase III Class III Promotor-AAV9, tRNA^{Tyr}-RNA polymerase III Class III Promotor-AAV9, tRNA^{His}-RNA polymerase III Class III Promotor-AAV9, tRNA^{Met}-RNA polymerase III Class III Promotor-AAV9, and tRNA^{Trp}-RNA polymerase III Class III Promotor-AAV9, such as tRNA^{Ala}-sRNA-AAV, tRNA^{Gly}-ssRNA-AAV, tRNA^{Tyr}-ssRNA-AAV, tRNA^{His}-ssRNA-AAV, tRNA^{Met}-ssRNA-AAV, and tRNA^{Trp}-ssNA-AAV, such as tRNA^{Ala}-sRNA-AAV9, tRNA^{Gly}-ssRNA-AAV9, tRNA^{Tyr}-sRNA-AAV9, tRNA^{His}-ssRNA-AAV9, tRNA^{Met}-ssRNA-AAV9, and tRNA^{Trp}-ssRNA-AAV9, such as tRNA^{Ala}-U6 ssRNA-AAV, tRNA^{Gly}-U6 ssRNA-AAV, tRNA^{Tyr}-U6 ssRNA-AAV, tRNA^{H}'s-U6 ssRNA-AAV, tRNA^{Met}-U6 ssRNA-AAV, and tRNA^{Trp}-U6 ssRNA-AAV, such as tRNA^{Ala}-U6 ssRNA-AAV9, tRNA^{Gly}-U6 ssRNA-AAV9, tRNA^{Tyr}-U6 ssRNA-AAV9, tRNA^{H}'s-U6 ssRNA-AAV9, tRNA^{Met}-U6 ssRNA-AAV9, and tRNA^{Trp}-U6 ssRNA-AAV9.

In an exemplary embodiment of the present compound the compound may be for overexpressing tRNA.

In an exemplary embodiment of the present compound overexpression may be established of tRNA^{Ala}, tRNA^{Gly}, tRNA^{Tyr}, tRNA^{His}, tRNA^{Met}, tRNA^{Trp}, and combinations thereof.

In an exemplary embodiment of the present compound the compound may be in the form of a viral vector, a synthetic tRNA, such as a chemical tRNA, and combinations thereof.

In an exemplary embodiment of the present compound the medicament may be for intrathecal application, for cerebral application, for the Peripheral Nervous System, for systemic application, and combinations thereof.

In an exemplary embodiment the present compound may be partially or fully embedded, such as embedded in a suitable carrier, such as in a lipid comprising carrier.

In a second aspect, the present invention relates to a dosage comprising a compound according to the invention.

In an exemplary embodiment of the present dosage the dosage may be a single dosage, or wherein the dosage may be a multiple dosage.

In a third aspect, the present invention relates to an in vivo or in vitro method of treating or preventing a heterozygous mutated cell, or preventing symptoms thereof, or for mitigating symptoms thereof, or for regeneration of impaired cells, or for gene therapy, or for RNA therapy, or a combination thereof.

In an exemplary embodiment of the present method the method may be repeated, such as 1-10 times, preferably repeated with intervals, such as regular intervals, such as with intervals of 1 month-12 months.

In a fourth aspect, the present invention relates to a method of introducing a cognate tRNA or tRNA encoding sequence into a heterozygous mutated cell.

In an exemplary embodiment of the present method the tRNA or tRNA encoding sequence may be obtained from a mammal.

In an exemplary embodiment of the present method the tRNA or tRNA encoding sequence may be natural or synthetic.

In an exemplary embodiment of the present method the tRNA may comprise an anticodon, such as a GCC anticodon.

The invention is further detailed by the accompanying figures and examples, which are exemplary and explanatory of nature and are not limiting the scope of the invention. To the person skilled in the art it may be clear that many variants, being obvious or not, may be conceivable falling within the scope of protection, defined by the present claims. In addition, reference is made to an article submitted for publication by Zuko, Storkebaum, et al entitled “tRNA overexpression rescues peripheral neuropathy caused by mutations in tRNA synthetase”, which document, and its contents, are hereby incorporated by reference.

### SUMMARY OF FIGURES

FIGS. 1, 2A-G, 3A_H, 4A-I, and 5A-I show details of the present invention.

### DETAILED DESCRIPTION OF FIGURES

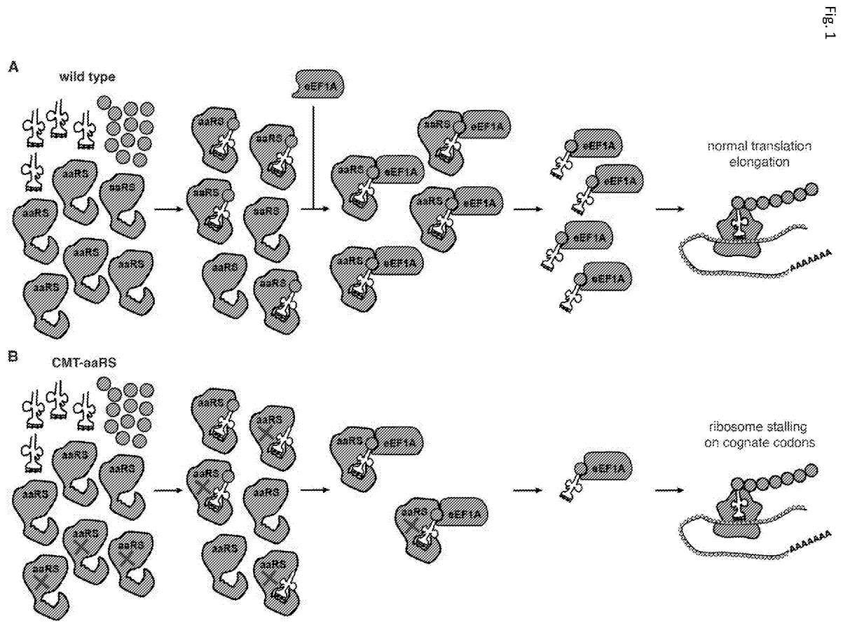

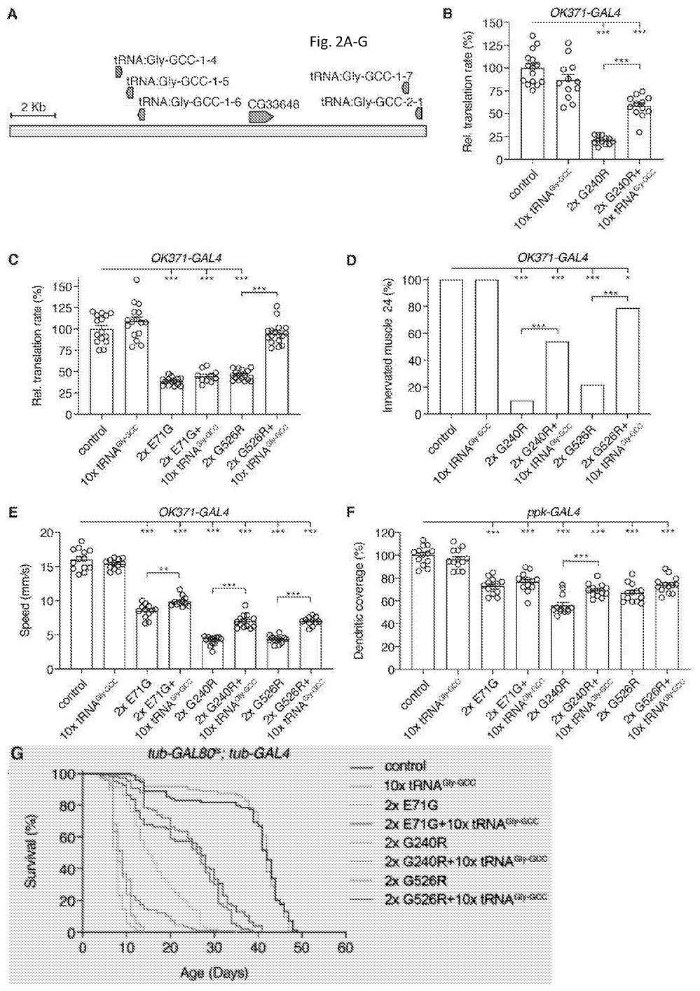

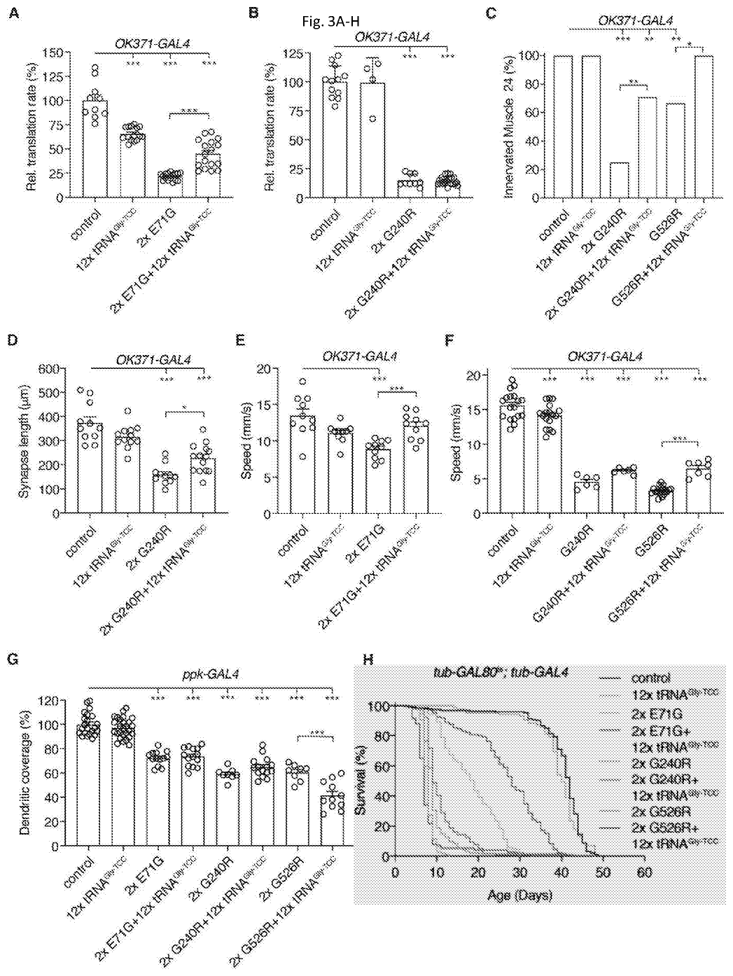

FIG. 1: Molecular mechanism underlying CMT-aaRS. (A) In the wild type situation, the tRNA synthetase (aaRS) binds the cognate tRNA and amino acid, activates the amino acid, and aminoacylates the tRNA. The aminoacylated (‘charged’) tRNA is transferred to the eukaryotic elongation factor 1A (eEF1A), which delivers the tRNA to the ribosome for use during translation elongation. (B) In CMT-aaRS, both wild type and CMT-mutant aaRSs are present, derived from the wild type and CMT-mutant AARS alleles, respectively. The CMT-mutant aaRS binds the cognate tRNA and possibly also the amino acid, may or may not activate the amino acid and aminoacylate the tRNA, but fails to release the tRNA or releases at a very slow pace. As a consequence, the cellular pool of the cognate tRNA is reduced under a critical threshold, and insufficient cognate tRNA is available for aminoacylation by the wild type aaRS. This results in insufficient supply of the aminoacylated tRNA to the ribosome, and stalling of the ribosome on cognate codons.

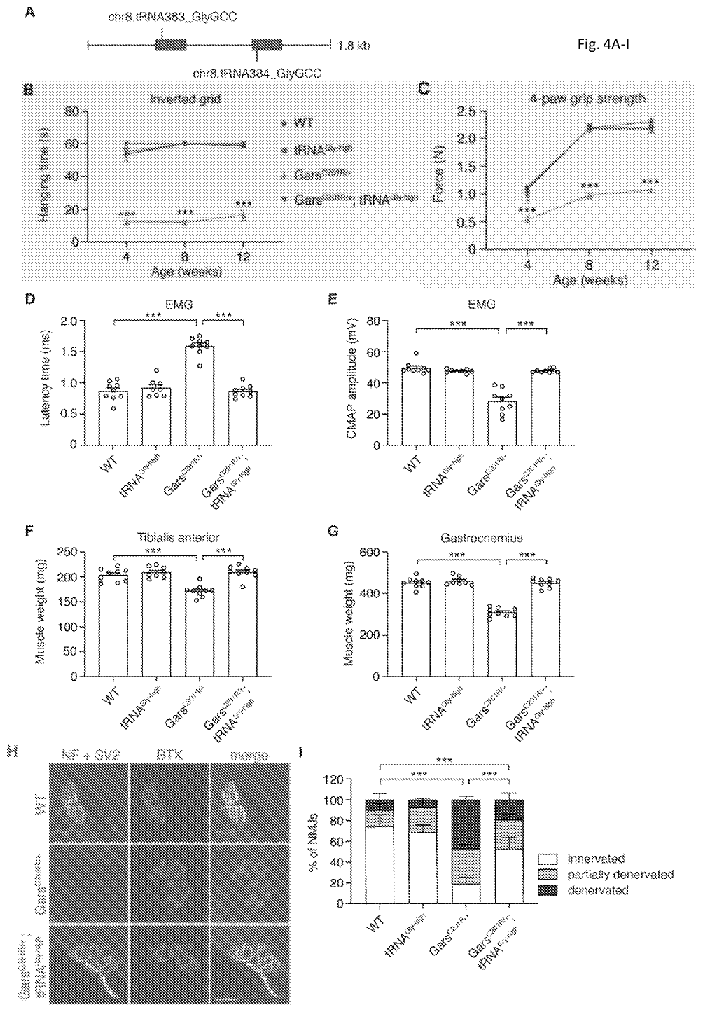

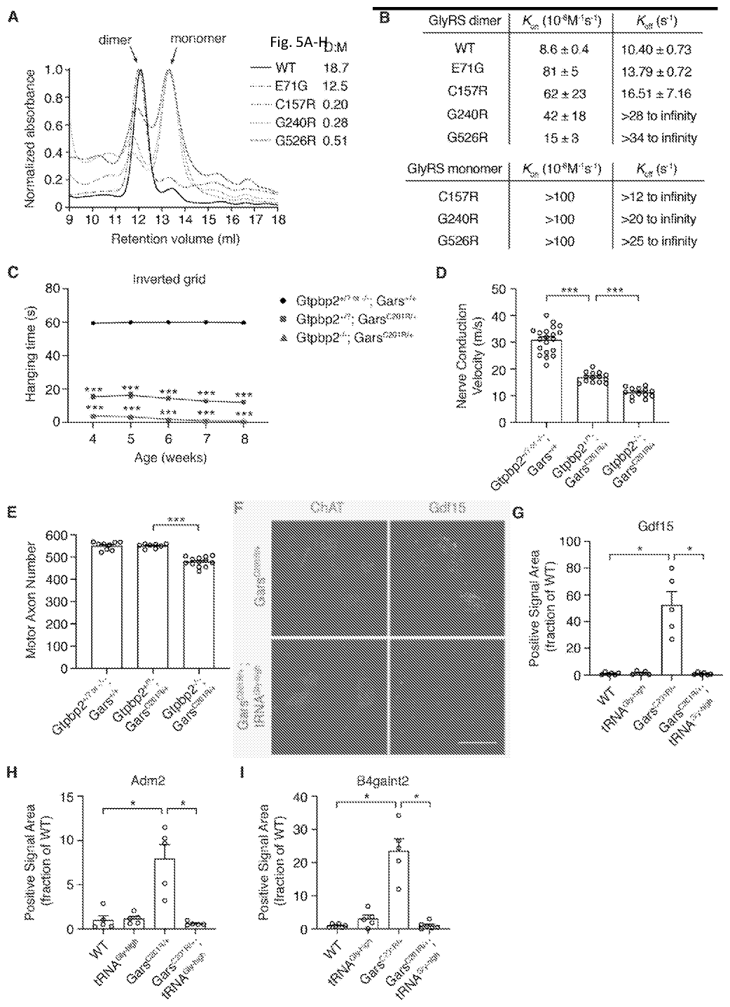

FIG. 2A-G: tRNA^{Gly-GCC} overexpression rescues inhibition of protein synthesis and peripheral neuropathy phenotypes in Drosophila CMT2D models. (A) Schematic overview 5 of the genomic region contained in the BAC used to generate tRNA^{Gly-GCC} transgenic Drosophila. (B,C) Relative translation rate (% of driver-only control) as determined by FUNCAT in motor neurons (OK371-GAL4) of larvae expressing G240R (B), E71G (C), or G526R (C) GlyRS mutants (2×: two transgene copies), in the presence or absence of the 10× tRNA^{Gly-GCC}transgene. n=10-17 animals per genotype; ***p<0.0001 by Brown-Forsythe and Welch ANOVA. (D) Percentage of larvae with innervated muscle 24. GlyRS transgenes were selectively expressed in motor neurons (OK371-GAL4), in the presence or absence of 10× tRNA ^{Gly-GCC} Control larvae are driver-only. n=19-26 animals per genotype; *p<0.05; ***p<0.005 by Chi-square test. (E) Climbing speed (mm/s) in an automated negative geotaxis assay of 7-day-old male flies that selectively express GlyRS transgenes in motor neurons (OK371-GAL4), in the presence or absence of 10× tRNA^{Gly-GCC}. Control flies are driver-only. n=13 groups of 10 flies per genotype; **p<0.01; * * *p<0.0001 by two-way ANOVA. (F) Dendritic coverage (% of driver-only control) of class IV multidendritic sensory neurons in the larval body wall upon selective expression of GlyRS transgenes in these sensory neurons (ppk-GAL4), in the presence or absence of 10× tRNA^{Gly-GCC}. n=13 animals per genotype; ***p<0.005 by two-way ANOVA. (G) Life span of flies ubiquitously expressing GlyRS transgenes from the adult stage onwards (tub-GAL80^{ts}; tub-GAL4), in the presence or absence of 10× tRNA^{Gly-GCC} Control flies are driver-only. n=79-126 flies per genotype; p<0.0001 for each GlyRS mutant versus GlyRS mutant+10× tRNA^{Gly-GCC} by Log-rank (Mantel-Cox) test.

FIG. 3A-H: tRNA^{Gly-TCC} overexpression rescues inhibition of protein synthesis and peripheral neuropathy phenotypes in Drosophila CMT2D models. (A,B) Relative translation rate (% of driver-only control) as determined by FUNCAT in motor neurons (OK371-GAL4) of larvae expressing E71G (A) or G240R (B) GlyRS mutants, in the presence or absence of the 12× tRNA^{Gly-TCC} transgene. n=10-17 (A) and 4-20 (B) animals per genotype; ***p<0.005 by Brown-Forsythe and Welch ANOVA. (C) Percentage of larvae with innervated muscle 24. G240R or G526R GlyRS was selectively expressed in motor neurons (OK371-GAL4), in the presence or absence of 12× tRNA^{Gly-TCC} Control flies are driver-only. n=12-27 animals per genotype; *p<0.05; **p<0.01; ***p<0.0001 by Chi-square test. (D) Synapse length on distal muscle 1/9 of larvae selectively expressing GlyRS _G240R in motor neurons (OK371-GAL4), in the presence or absence of 12× tRNA^{Gly-TCC}. Control flies are driver-only. n=11-14 animals per genotype; *p<0.05; ***p<0.0001 by one-way ANOVA. (E,F) Climbing speed (mm/s) of 7-day-old male flies. E71G (E), G240R (F), or G526R (F) GlyRS was selectively expressed in motor neurons (OK371-GAL4), in the presence or absence of 12× tRNA^{Gly-TCC}. Control flies are driver-only. n=6-19 groups of 10 flies per genotype; ***p<0.005 by Brown-Forsythe and Welch ANOVA. (G) Dendritic coverage (% of driver-only control) of class IV multidendritic sensory neurons, in which GlyRS transgenes were selectively expressed (ppk-GAL4), in the presence or absence of 12× tRNA^{Gly}^{-TCC}. n=8-15 animals per genotype; ***p<0.0001 by one-way ANOVA. (H) Life span of flies ubiquitously expressing GlyRS transgenes from the adult stage onwards (GAL80^{ts}; tub-GAL4), in the presence or absence of 12× tRNA^{Gly-TCC}. Control flies are driver-only. n=85-193 flies per genotype; p<0.0001 for each GlyRS mutant versus GlyRS mutant+12× tRNA^{Gly-TCC} by Log-rank (Mantel-Cox) test.

FIG. 4A-I: tRNA^{Gly-GCC} overexpression rescues peripheral neuropathy in Gars^{C201R/+} mice. (A) Schematic overview of the mouse genomic fragment used for generation of tRNA^{Gly-GCC} transgenic mice. (B) Hanging time in the inverted grid test of a cohort of male WT, tRNA^{Gly-high}, Gars^{C201R/+}, and Gars^{C201R/+}; tRNA^{Gly-high} littermate mice at 4, 8, and 12 weeks of age. n=8-9 mice per genotype; ***p<0.0005 by one-sample t-test (theoretical mean of WT) and two-tailed unpaired t-test with Bonferroni's multiple comparisons test per time point. (C) 4-paw grip strength of the same cohort of mice as measured by dynamometer. n=8-9 mice per genotype; ***p<0.001 by two-way ANOVA with Tukey's multiple comparisons test per time point. (D,E) l Analysis of the same cohort of mice at 12 weeks of age by electromyography (EMG). (D) Latency time between stimulation of the sciatic nerve at the sciatic notch level and detection of a compound muscle action potential (CMAP) in the gastrocnemius muscle. n=8-9 mice per genotype; ***p<0.0001 by two-way ANOVA with Tukey's multiple comparisons test. (E) CMAP amplitude in the gastrocnemius muscle. n=8-9 mice per genotype; ***p<0.0005 by Brown-Forsythe and Welch ANOVA. (F,G) Weight of the tibialis anterior (F) and gastrocnemius (G) muscles of the same cohort of mice at 12 weeks of age. n=8-9 mice per genotype; ***p<0.0001 by two-way ANOVA with Tukey's multiple comparisons test. (H,I) Representative images (H) and quantification (I) of NMJ innervation status in the plantaris muscle. In (H), the presynaptic nerve ending was visualized by immunostaining for neurofilament (NF) and SV2, while postsynaptic acetylcholine receptors were visualized by TRITC-conjugated bungarotoxin (BTX). n=5 mice per genotype; ***p<5×10⁻⁶ by Fisher's Exact test. Scale bar: 25 μm.

FIG. 5A-I: Mechanism underlying rescue of CMT2D phenotypes by tRNA^{Gly} overexpression. (A) Size-exclusion chromatography of purified recombinant human GlyRS proteins reveals different partitioning between dimer and monomer forms of WT, E71G, C157R (equivalent to mouse C201R), G240R and G526R variants. Dimer:monomer (D:M) ratio of each GlyRS variant is indicated. (B) Kₒₙ and K_{off} values of Drosophila tRNA^{Gly-GCC} binding and release to the indicated human GlyRS variants. Kₒₙ and K_{off} values are shown for dimer and monomer fractions. (C) Hanging time in the inverted grid test of male Gtpbp2^{+/? or -/-}; Gars^{+/+} (control), Gtpbp2^{+/?}; Gars^{C201R/+}, and Gtpbp2^{-/-}; Gars^{C201R/+} littermate mice at 4, 5, 6, 7 and 8 weeks of age. n=15-28 mice per genotype group; ***p<0.0005 by one-sample t-test (theoretical mean of Gtpbp2^{+/? or -/-}, Gars^{+/+}, and two-tailed unpaired t-test with Bonferroni's multiple comparisons test per time point. (D) Nerve conduction velocity of the sciatic nerve of Gtpbp2^{+/? or -/-}; Gars^{+/+}, Gtpbp2^{+/?}; Gars^{C201R/+}, and Gtpbp2^{-/-}; Gars^{C201R/+} littermate mice at 8 weeks of age. n=13-20 mice per genotype group; ***p<0.0001 by Brown-Forsythe and Welch ANOVA. (E) Axon number in the motor branch of the femoral nerve Gtpbp2^{+/? or -/-}; Gars^{+/+}, Gtpbp2^{+/?}; Gars^{C201R/+}, and Gtpbp2^{-/-}; Gars^{C201R/+} littermate mice at 8 weeks of age. n=8-13 per genotype group; ***p<0.0001 by one-way ANOVA. (F-I) Representative images (F) and quantification of fluorescent in situ hybridization for the ATF4 target genes Gdf15 (G), Adm2 (H), and B4galnt2 (I). Scale bar: 50 μm. n=5-6 mice per genotype; ***p<0.05 by two-tailed Welch's t-test with Bonferroni's multiple comparisons correction.

The figures are further detailed in the description.

### EXAMPLES/EXPERIMENTS

Inventors generated Drosophila models for CMT-TyrRS and CMT-GlyRS, which recapitulate several hallmarks of the human disease. Loss of aminoacylation activity is not a common feature of CMT-mutant aaRSs and thus considered not to be required to cause CMT. Furthermore, a novel method which allows to cell-type-specifically monitor translation in Drosophila in vivo was developed. This ground-breaking approach revealed that each of six distinct GlyRS or TyrRS mutants substantially reduced global protein synthesis in motor and sensory neurons. Based on these unprecedented novel insights, it is considered that impaired translation constitutes a common pathogenic mechanism underlying CMT-aaRS. It is found that, strikingly, transgenic overexpression of tRNAGly in Drosophila CMT-GlyRS models fully rescued translation and partially but substantially rescued peripheral neuropathy phenotypes. Consistently, generation of tRNAGly overexpressing mice revealed that tRNAGly overexpression fully prevented peripheral neuropathy in a CMT-GlyRS mouse model. Finally, overexpression of Drosophila orthologs of the elongation factor eEF1A partially but significantly rescued peripheral neuropathy in Drosophila CMT-GlyRS models. Therefore, it is considered that CMT-mutant aaRSs bind the cognate tRNA, may or may not aminoacylate it, but fail to transfer the aminoacylated tRNA to eEF1A. Consequently, the supply of aminoacylated cognate tRNA to the ribosome may drop below a critical threshold, causing the ribosome to pause or stall on cognate codons, thus explaining the translation defect (FIG. 1).

Results

It is found that tRNA^{Gly} overexpression rescues peripheral neuropathy in Gars^{C201/R+} mice. Also, tRNA^{Tyr}overexpression rescued peripheral neuropathy in CMT-TyrRS Drosophila models. Further tRNA^{Tyr} overexpressing mice may rescue peripheral neuropathy in a CMT-TyrRS mouse model. Also, translation may be inhibited in motor neurons of CMT-GlyRS and CMT-TyrRS mice. It is found that translation elongation is affected in CMT-GlyRS mice. The degree of phenotypic rescue is found to be dependent on the level of tRNAGly overexpression. tRNAGly overexpression induced full rescue of peripheral neuropathy in CMT-GlyRS mice versus partial rescue in Drosophila. Northern blotting revealed substantial tRNAGly overexpression in mice versus moderate overexpression in Drosophila. Drosophila lines with higher tRNAGly overexpression are generated to evaluate whether this results in more substantial/full rescue of peripheral neuropathy. It is found that only overexpression of cognate tRNA can rescue. It is confirmed that tRNAGly overexpression does not rescue CMT-TyrRS models and that tRNATyr overexpression does not rescue CMT-GlyRS models. Furthermore, tRNAGly with GCC anticodon rescued CMT-GlyRS models. Ribosome profiling/foot printing on spinal cord extracts from CMT-GlyRS and CMT-TyrRS mice is performed to detect ribosome stalling on Gly or Tyr codons, respectively.

For all above mentioned compounds similar results are achieved or found.

The invention although described in detailed explanatory context may be best understood in conjunction with the accompanying examples and figures as detailed above.

Some exemplary qualifications and quantifications are given below.

1. A compound for overexpression of cognate tRNA for use in a medicament for treating a heterozygous mutated cell, wherein the compound comprises a transfer RNA, and a vector, wherein the vector is coupled to the wherein the tRNA is selected from tRNA^{Ala}, tRNA^{Gly}, tRNA^{Tyr}, tRNA^{His}, tRNA^{Met}, tRNA^{Trp}, and combinations thereof.

2. The compound according to claim 1, wherein the heterozygous mutated cell is a neuron.

3. The compound according to claim 2, wherein the peripheral neuropathy is selected from an inherited neuromuscular disorder, a central nervous system disorder, a brain disorder, a motoric nerve disorder, a sensory nerve disorder, or a combination thereof.

4. The compound according to claim 1, wherein the vector is an adeno-associated viral (AAV) vector, preferably an AAV9 (serotype 9) vector.

5. The compound according to claim 1, wherein the promotor is an RNA polymerase III promotor.

6. The compound according to claim 1, wherein the compound is for overexpressing tRNA.

7. Compound according to claim 1, wherein the compound is in a form selected from a viral vector, a synthetic tRNA, and combinations thereof.

8. The compound according to claim 1, wherein the medicament is for an application selected from intrathecal application, cerebral application, the Peripheral Nervous System, for systemic application, and combinations thereof.

9. The compound according to claim 1, wherein the compound is selected froni partially embedded and fully embedded.

10. A dosage comprising a compound according to claim 1, wherein in a viral gene transfer the compound comprises >10¹² vg/kg body mass.

11. The dosage according to claim 10, wherein the dosage is selected from a single dosage, and a multiple dosage.

12. An method selected from an iln vivo method and an in vitro method of treating a heterozygous mutated cell, of preventing a heterozygous mutated cell, of preventing symptoms thereof, of mitigating symptoms thereof, of regeneration of impaired cells, of gene therapy, of RNA therapy, and a combination thereof, comprising
providing a dosage according to claim 10, and
applying the dosage, wherein applying is selected from intrathecal application, from cerebral application, from application to the Peripheral Nervous System, or and from systemic application.

13. The method according to claim 12, wherein the method is repeated.

14. A method of introducing a sequence selected from a cognate tRNA and tRNA encoding sequence into a heterozygous mutated cell, comprising providing the heterozygous mutated cell,
providing the tRNA or tRNA encoding sequence in a suitable form, wherein the tRNA or tRNA encoding sequence is selected from tRNA^{Ala}, tRNA^{Gly}, tRNA^{Tyr}, tRNA^{His}, tRNA^{Met}, tRNA^{Trp}, and combinations thereof, and
introducing the tRNA or tRNA encoding sequence into the heterozygous mutated cell.

15. The method according to claim 14, wherein the tRNA or tRNA encoding sequence is obtained from a mammal.

16. The method according to claim 14, wherein the tRNA or tRNA encoding sequence is selected from a natural or-sequence and a synthetic sequence.

17. The method according to claim 14, wherein the tRNA comprises an anticodon.

18. The compound according to claim 1, wherein the compound comprises a promotor, wherein the vector is coupled to the tRNA promotor, and wherein the promotor is coupled to the tRNA.

19. The compound according to claim 2, wherein the heterozygous mutated cell is selected from a motor neuron and a sensory neuron.

20. The compound according to claim 3, wherein the peripheral neuropathy is Charcot-Marie-Tooth peripheral neuropathy.
